(19) United States
(12) Patent Application Publication
(10) Pub. No.: US 2023/0240213 A1
(43) Pub. Date: Aug. 3, 2023

(54) METHOD FOR IMPROVING SOYBEAN HYBRID YIELD

(71) Applicant: Anhui Agricultural University, Hefei City (CN)

(72) Inventors: Xiaobo WANG, Hefei City (CN); Duo ZHAO, Hefei City (CN); Jiajia LI, Hefei City (CN); Yan ZHANG, Hefei City (CN); Jiale LIU, Hefei City (CN); Huimin ZHAI, Hefei City (CN)

(21) Appl. No.: 17/874,409

(22) Filed: Jul. 27, 2022

(30) Foreign Application Priority Data
Dec. 31, 2021 (CN) 202111665606.3

(51) Int. Cl. A01H 1/02; A01H 6/54

(52) U.S. Cl. CPC A01H 1/02; A01H 6/542

(57) ABSTRACT

The disclosure relates to the technical field of soybean breeding, in particular to a method for improving soybean hybrid yield. In the method, fertile soybean is taken as a male parent, and pollen from the male parent is prepared into a pollen compound solution, which is carried with a spraying device to be directionally spayed to a female parent for hybridization, where the female parent is a genic male sterile soybean plant. By preparation of an appropriate proportion of pollen compound solution and pollination assisted by artificial spraying, the disclosure may not only enhance the pollen activity, but also improve the pollen utilization rate and greatly improve the efficiency of soybean hybrid production, thereby improving the soybean yield. It effectively saves human resources, improves the efficiency of soybean hybrid production, and meets the needs of soybean industry development.

### CROSS REFERENCE TO RELATED APPLICATION

This patent application claims the benefit and priority of Chinese Patent Application No. 202111665606.3, entitled “Method For Improving Soybean Hybrid Yield” filed on Dec. 31, 2021, the disclosure of which is incorporated by reference herein in its entirety as part of the present application.

### TECHNICAL FIELD

The disclosure relates to soybean breeding, and particularly to a method for improving soybean hybrid yield.

### BACKGROUND ART

Crop heterosis is a crucial biological phenomenon, taking advantage of which is one of the important breeding methods of soybean. That is to say, the hybrid generation after the hybridization of two organisms with different genetic compositions is superior to the parent in terms of growth potential, viability, stress resistance, yield and quality, and meets the production requirements. However, the characters of the first generation of hybrids are more complex, and the methods that utilize heterosis are different due to the differences in pollination, reproduction and genetic characteristics of crops.

Soybean is a natural self-pollinating crop, in which the incidence of natural outcrossing is only 0.4%. It is also a hermaphroditic plant with small flower organs. Generally, the seed setting rate by artificial-assisted pollination is low. Moreover, soybeans are easy to drop flowers and pods, with a general dropping rate of flower and pod each up to about 45% to 70%, even to 70% to 80%, resulting in a low reproduction coefficient. Under this circumstance, traditional hybridization methods for soybean planting can hardly meet the large demand for progeny with specific traits in production and daily life, and there are problems such as difficulty in utilizing heterosis, low economic benefits, and a waste of manpower and material resources.

### SUMMARY

In order to solve the above technical problems, the present disclosure provides a method for improving efficiency of soybean hybrid production.

The present disclosure adopts the following technical solutions:
The method includes collecting pollen of a fertile soybean as a male parent at a full flowering stage to prepare a pollen compound solution, and spraying the pollen compound solution directionally on a female parent for hybridization, in which the female parent is a genic male sterile (GMS) soybean plant;
the pollen compound solution is formulated as follows: mixing the collected pollen and deionized water evenly to prepare a pollen solution of 3 to 10 mg/mL, then adding and mixing a surfactant, a pheromone and the pollen liquid in a volume ratio of (1-5) : (1-5): (90-98) evenly to obtain the pollen compound solution.

In some embodiments, the GMS soybean plant is a plant where a male sterile gene fragment on chromosome 13 is deleted.

In some embodiments, the surfactant is lauramidopropyl betaine A-20AB; the pheromone is a mixture of queen bee pheromone, honeycomb and nutrients required by a plant in a volume ratio of 49:49:2, and the nutrients required by a plant includes nitrogen, phosphorus, potassium, boron, and molybdenum in a mass ratio of 3:3:2:1:1.

In some embodiments, the surfactant, the pheromone and the pollen solution are mixed evenly in a volume ratio of 1:1:98.

In some embodiments, the pollen compound solution is added with hydroxypropyl methyl cellulose (HPMC) at 7 g per 100 mL of the pollen compound solution.

In some embodiments, the method includes the following steps:
S1. sowing desired soybean seeds of the male parent and the female parent successively in two fields separately by interval sowing, and performing field management, respectively;
S2. at a full flowing stage of the male parent, collecting pollen of the male parent and storing the pollen in a refrigerator at 4° C.;
S3. at a full flowing stage of the female parent, preparing the pollen in S2 into the pollen compound solution, and directionally spraying to the female parent for pollination with a spraying device;
S4. at an interval of 1 to 3 days after a first pollination, re-pollinating once by the spraying; and
S5. performing the field management until the female parent is mature and harvested.

In step S1, the male parent is a fertile soybean with an average number of 80 to 100 flowers per plant and an amount of soybean pollen greater than 3800 grains per flower, and the male and female parents are sowed at 18,000 to 25,000 plants/667 m².

In some embodiments, in step S1, the male parent are sowed firstly, and then the female parent after a two-day interval to make full bloom stage of the male parent 2 to 3 days earlier than that of the female parent.

In some embodiments, the full flowering stage is when soybean flowering plants account for at least ⅔ of all plants.

In some embodiments, in step S3, the spraying is conducted at 7 to 9 a.m. for 10-20 min/666.7 m², with the pollen compound solution being 3.8 to 4.5 L/666.7 m².

The beneficial effects of embodiments of the present disclosure include the followings.

1. This technology selects soybean varieties with more excellent traits, large pollen amount and high combining ability as the female parent, and soybean varieties with more complementary excellent traits, suitability for local planting, different geographical origins, good combining ability with the female parent, and high yield as the female parent for hybridization, which overcomes the problems in conventional hybrid breeding that soybean is a natural self-pollinating crop with a low outcrossing rate low rate, and its flower is hermaphroditic and small; the reproduction coefficient and seed setting rate by artificial-assisted pollination are low; manpower and material resources are wasted and utilization of heterosis is hard. At the same time, the female parent belongs to the male sterile line population, ensuring the parental purity of the soybean plants.

2. Surfactant, pheromone and pollen are used to prepare the pollen compound solution, in which the surfactant enhances the viability of soybean pollen, with excellent affinity to flowers, and will not damage the pollen stigma; the component pheromone contains signals to attract bees, honeycomb and nutrients required by plants such as nitrogen, phosphorus, potassium, boron, molybdenum and so on, which has a special smell to attract insects, improves natural pollination efficiency, increases yield and contributes to pollination evenness and fruit uniformity. The pheromone concentration used in this application is sufficient to attract bees, butterflies, etc. near soybean plants to pollinate soybean, improves the pollination rate of insect pollinators and the outcross rate of sterile lines, and also protects flowers and pods to a certain extent. The additional addition of HPMC enhances the vitality of pollen and maintains the stability of the pollen compound solution.

3. Preparing a suitable pollen compound solution and assisting pollination with spraying devices not only enhance the pollen activity, but also improve the pollen utilization rate and greatly improve the efficiency of soybean hybrid production, thereby increasing the soybean yield. It effectively saves human resources, improves the efficiency of soybean hybrid production, and meets the needs of soybean industry development.

### BRIEF DESCRIPTION OF THE DRAWINGS

FIG. 1 is three pictures labeled as a, b, and c, wherein a is a picture of soybean podding of the treatment group in Example 3; b is a picture of soybean podding of the control group in Example 3, and for the purpose of observation, the soybean leaves part are cut off; and c is a comparison of typical soybean podding between the treatment group and the control group in Example 3, where the left side refers to the treatment group, and the right side refers to the control group.

### DETAILED DESCRIPTION OF THE EMBODIMENTS

For ease of understanding, the technical solutions of the present disclosure will be described in more detail below with reference to the examples and the accompanying drawings.

### Example 1

A method for improving efficiency of soybean hybrid production, the steps of which were as follows:

S0. Preliminary preparation:

S01. Selection of male and female parents: a soybean variety with various excellent traits, large pollen amount, and high combining ability and undergone male sterile gene treatment is selected as a female parent; a soybean varieties with a different geographical origin from that of the female parent, high combining ability and high yield is chosen as the male parent; for high yield, it is required that the male parent has large flowers and a large amount of pollen, with an average number of 80 to 100 flowers per plant and an amount of soybean pollen greater than 3800 grains per flower.

In the male sterile gene treatment, the gene editing technology CRISPR/Cas9 is used to cut the male sterile gene fragment on chromosome 13 of soybean to obtain a GMS plant with a deletion of the male sterile gene fragment..

S02. Land preparation: a plot with flat terrain, good fertility, convenient irrigation and drainage, consistent previous crops, and preferably no soybean plantation for at least two years is selected, and weeding and land leveling are performed.

S1. The desired soybean seeds of the female parent and male parent are broadcast separately in stages in the plot selected in S02, and the sowing of the female parent is conducted two days later than that of the male parent to make the full flowering stage of the female parent 2 to 3 days later than that of the male parent. The protection row is set up in the plot, and field management is carried out respectively; the density of broadcasting is 180,000 to 250,000 plants/667 m², and the density for the protection row is 54.7 /m².

The filed management is conducted as follows: herbicides is sprayed for sealing after the sowing, the seedlings are thinned during the seedling period, and weeds are weeded manually once upon emergence. Insecticides are not used in the whole flowering stage to facilitate pollination by insects, thereby increasing the natural outcrossing rate of soybean male sterile material.

S2. During the flowering period of the male parent, the anthers are taken manually or mechanically at the full flowering stage, and irradiated with electric bulbs or placed in a constant temperature drying box to disperse and collect the pollen, and then the collected pollen were stored in a refrigerator at 4° C. for later use.

S3. During the flowering period of the female parent, that is, when the flowering plants account for at least ⅔ of the total plants, the pollen in S2 is prepared into pollen compound solution; the pollen compound solution is formulated as follows: the collected pollen and deionized water are mixed evenly to formulate a 3 to 10 mg/mL of pollen solution, and then a surfactant, a pheromone and the pollen solution are mixed evenly according to the volume ratio of (1-5):(1-5):(90-98) to obtain the pollen compound solution; 7 g of HPMC is added to each 100 mL of pollen compound solution to enhance the pollen viability.

The surfactant is lauramidopropyl betaine A-20AB; the pheromone is a mixture of queen bee pheromone, honeycomb and nutrient elements required by plants according to a volume ratio of 49:49:2, and the nutrient elements required by the plants includes nitrogen, phosphorus, potassium, boron and molybdenum in a mass ratio of 3:3:2:1:1.

The prepared pollen compound solution is poured into the spraying device for directional spraying and pollination of the female parent. The spraying is conducted at 7 to 9 a.m. with a desired spraying device that is suitable for the size of soybean flower. The pollen compound solution is put into the spraying device and shaken to evenness, and the solution is directionally bombarded on the male sterile stigma in the form of a tiny aerosol, so as to complete the pollination process. An amount of the pollen compound solution per 666.7 m² of field is 3.8-4.5 L, and the bombardment lasts 10 to 20 min/666.7 m².

S4. After an interval of 1 to 3 days of a first pollination, the same method is repeated once for re-pollination, so as to improve the soybean outcrossing pollination rate.

S5. The field management is continued until the female parent pods are mature, which are then harvested in batches.

### Example 2

A soybean hybrid production test was carried out at Dayang Town, Hefei, Anhui Province, in accordance with the method of Example 1, and the planting method was as follows:

1. Variety selection: the soybean variety Wanhuang 506 suitable for summer planting in Huaibei soybean production area in Anhui Province was selected, and it was mixed planted with the male sterile material, with a mixed weight ratio of 5:1.

2. Selection of experimental plots: the planting plots were two plots in the north and south with similar soil fertility and many natural wild bees and other pollinators, and rice was planted as a previous crop.

3. Land preparation: in the first ten days of July, after the soil is ploughed and raked, the row was driven at a row spacing of 45 cm;

4. Sowing: the mixed soybean seeds were broadcast at a density of 22,800 plants/667 m²;

5. Field management: herbicide was sprayed for sealing after the sowing, the seedlings was not thinned during the seedling stage, weeding was conducted upon emergence of weeds, and insectcide was not done during the flowering stage;

6. pollination: outcrossing pollination was carried out to the soybean plants in the northern plot as a treatment group, the outcrossing pollination method was carried out by steps S2-S4 in Example 1; the southern plot was not treated, and set as a control group;

7. Harvesting: after the soybeans were mature, the seeds on the sterile plants were harvested in time for drying. Due to the inconsistent seed maturity of sterile plants, the seeds were harvested in batches to ensure seed quality.

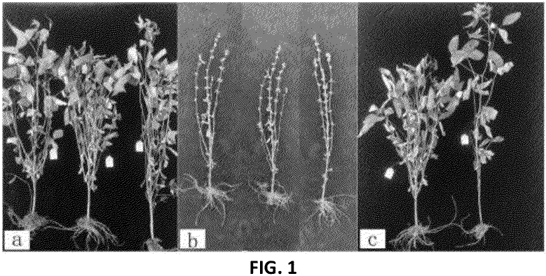

After pollination of step S6, 30 male sterile lines were randomly selected from the control group and the treatment group respectively for statistics. Podding results are shown in Tables 2 and 3, ant typical podding in the control group and the treatment group are shown in FIG. 1.

TABLE 2

Podding Results

| Group | Pods per plant | | | | | | | | | | Average pods |
|---|---|---|---|---|---|---|---|---|---|---|---|
| Treatment group | 20 | 20 | 13 | 10 | 13 | 17 | 19 | 26 | 20 | 16 | 19.4 |
| | 19 | 20 | 25 | 11 | 20 | 34 | 19 | 18 | 12 | 11 | |
| | 15 | 13 | 27 | 17 | 22 | 30 | 20 | 42 | 13 | 19 | |
| Control group | 5 | 10 | 3 | 5 | 2 | 3 | 9 | 10 | 6 | 10 | 8.2 |
| | 14 | 9 | 12 | 13 | 8 | 5 | 6 | 7 | 9 | 5 | |
| | 13 | 3 | 9 | 7 | 9 | 6 | 10 | 9 | 15 | 13 | |

TABLE 3

Proportion of plants with more than 10 pods

| Group | Proportion of plants with more than 10 pods |
|---|---|
| Treatment group | 100% |
| Control group | 33% |

It could be seen from the test that the pod setting rate of the treatment group was significantly higher than that of the control group, the number of pods in the treatment group was more than 10, the average number of pods was 19, and the number of pods in a few sterile plants reached more than 30.

According to the present disclosure, the soybean pollen compound solution is prepared, and then applied to the stigma of the male sterile line plant by bombardment with drones, thereby completing the process of outcrossing and pollination. The method may greatly improve the efficiency of soybean hybrid seed production as well as the outcrossing rate of the soybean GMS plants, and is promising in application.

The above embodiments are only used to illustrate the technical solutions of the present disclosure, but not to limit the present disclosure; although the present disclosure has been described in detail with reference to the foregoing embodiments, those of ordinary skill in the art should understand that any modifications, equivalent replacements and improvements made within the principles shall be included within the protection scope of the present disclosure.

1. A method for improving efficiency of soybean hybrid production, comprising collecting pollen of a fertile soybean as a male parent at a flowering stage to prepare pollen compound solution, and spraying the pollen compound solution directionally to a female parent for hybridization, wherein the female parent is a genic male sterile (GMS) soybean plant;
the pollen compound liquid is formulated as follows: mixing the collected pollen and deionized water evenly to prepare a pollen solution of 3 to 10 mg/mL, and then adding and mixing a surfactant, a pheromone and the pollen liquid in a volume ratio of (1-5) : (1-5): (90-98) evenly to obtain the pollen compound solution.

2. The method for improving efficiency of soybean hybrid production according to claim 1, wherein, the GMS soybean plant is a plant in which a male sterile gene fragment on chromosome 13 is deleted.

3. The method for improving efficiency of soybean hybrid production according to claim 1, wherein, the pheromone is a mixture of queen bee pheromone, honeycomb and nutrients required by a plant in a volume ratio of 49:49:2, and the nutrients required by a plant comprises nitrogen, phosphorus, potassium, boron, and molybdenum in a mass ratio of 3:3:2:1:1.

4. The method for improving efficiency of soybean hybrid production according to claim 1, wherein, the surfactant, the pheromone and the pollen solution are mixed evenly in a volume ratio of 1:1:98.

5. The method for improving efficiency of soybean hybrid production according to claim 4, wherein, the pollen compound solution is further added with hydroxypropyl methyl cellulose (HPMC) at 7 g per 100 mL of the pollen compound solution.

6. The method for improving efficiency of soybean hybrid production according to claim 1, wherein the method comprises:
S1. sowing soybean seeds of the male parent and the female parent successively in two fields separately by interval sowing, and performing field management, respectively;
S2. at a full flowing stage of the male parent, collecting pollen of the male parent and storing the pollen in a refrigerator at 4° C.;
S3. at a full flowing stage of the female parent, preparing the pollen in S2 into the pollen compound solution, and directionally spraying to the female parent for pollination with a spraying device;
S4. at an interval of 1 to 3 days after a first pollination, re-pollinating once by the spraying; and
S5. performing the field management until the female parent is mature and harvested.

7. The method for improving efficiency of soybean hybrid production according to claim 6, wherein, in step S1, the male parent is a fertile soybean with an average number of 80 to 100 flowers per plant and an amount of soybean pollen greater than 3800 grains per flower, and the male and female parents are sowed at 18,000 to 25,000 plants/667 m².

8. The method for improving efficiency of soybean hybrid production according to claim 6, wherein, in step S1, the male parent are sowed firstly, and then the female parent after a two-day interval to make full bloom stage of the male parent 2 to 3 days earlier than that of the female parent.

9. The method for improving efficiency of soybean hybrid production according to claim 6, wherein, the full flowering stage is when soybean flowering plants account for at least ⅔ of all plants.

10. The method for improving efficiency of soybean hybrid production according to claim 6, wherein, in step S3, the spraying is conducted at 7 to 9 a.m. for 10 to 20 min/666.7 m², with the pollen compound solution being 3.8 to 4.5 L/666.7 m².

11. The method for improving efficiency of soybean hybrid production according to claim 6, wherein the GMS soybean plant is a plant in which a male sterile gene fragment on chromosome 13 is deleted.

12. The method for improving efficiency of soybean hybrid production according to claim 6, the pheromone is a mixture of queen bee pheromone, honeycomb and nutrients required by a plant in a volume ratio of 49:49:2, and the nutrients required by a plant comprises nitrogen, phosphorus, potassium, boron, and molybdenum in a mass ratio of 3:3:2:1:1.

13. The method for improving efficiency of soybean hybrid production according to claim 6, the surfactant, the pheromone and the pollen solution are mixed evenly in a volume ratio of 1:1:98.

14. The method for improving efficiency of soybean hybrid production according to claim 6, the pollen compound solution is further added with HPMC at 7 g per 100 mL of the pollen compound solution.
